(19) United States
(12) Patent Application Publication
(10) Pub. No.: US 2013/0237826 A1
(43) Pub. Date: Sep. 12, 2013

(54) PRECISION ULTRASONIC SCANNER FOR BODY PARTS WITH EXTENDED IMAGING DEPTH

(71) Applicant: Andrew K. Levien, Morrison, CO (US)

(72) Inventor: Andrew K. Levien, Morrison, CO (US)

(73) Assignee: ArcScan, Inc., Golden, CO (US)

(21) Appl. No.: 13/796,931

(22) Filed: Mar. 12, 2013

Related U.S. Application Data
(60) Provisional application No. 61/609,626, filed on Mar. 12, 2012.
(60) Provisional application No. 61/611,903, filed on Mar. 16, 2012.

(51) Int. Cl. A61B 8/00

(52) U.S. Cl. CPC A61B 8/4427
USPC 600/448; 600/459

(57) ABSTRACT

A method and apparatus are disclosed for performing an ultrasound scan on a body part and specifically for a portable instrument which directly attaches to the surface of the body. This apparatus provides very high resolution images and greatly increased depth of imaging for high resolution ultrasound of targeted subsurface body tissues with ultrasound image resolution far superior to that of known state of the art ultrasound instruments. Targeted tissues could include but not be limited to joints, ocular structures, and internal organs.

The method and apparatus disclosed are also directed towards providing an ultrasound imaging system that can produce high image resolution (down to about 100 μm) at depths approaching about 50 mm to about 60 mm. The method and apparatus can stabilize and provide accurate determination of the position of the body part relative to the ultrasound probe.

### CROSS-REFERENCE TO RELATED APPLICATIONS

The present application claims the benefits, under 35 U.S.C. §119(e), of U.S. Provisional Application Ser. No.61/609,626 entitled “Ultrasonic Scanner for Body Parts” filed Mar. 12, 2012 and U.S. Provisional Application Ser. No. 61/611,903 entitled “Extension of Imaging Depth for Ultrasonic Scanner” filed Mar. 16, 2012, both of which are incorporated herein by this reference.

### FIELD

The present invention relates in general to a method and apparatus for performing an ultrasound scan on a body part and specifically to a portable instrument which directly attaches to the surface of the body yet provides very high resolution images and greatly increased depth of imaging for high resolution ultrasound of targeted subsurface body tissues with ultrasound image resolution far superior to that of known state of the art ultrasound instruments.

### BACKGROUND

A challenge for any medical imaging system is to provide the highest possible image resolution while also attaining a high depth of image at a reasonable cost. Optical systems such as optical coherence tomography are compact and cost effective and provide excellent resolution. However, they are only capable of imaging a few millimeters into any opaque tissue surface as the light is rapidly absorbed. Current ultrasound systems are very compact and cost effective and have high tissue penetration depths of 100 mm or more. However, they offer relatively low resolution due to their low range of operating frequencies from about 5 MHz to about 10 MHz. MRI systems are well-known imaging systems that provide both high depth of image and high resolution. However, they are characterized by high cost, large size and a costly dedicated infrastructure. High frequency ultrasound systems (from about 20 MHz to about 80 MHz) can provide high resolution but only with a limited image depth.

There remains a need for a low cost, portable ultrasound imaging system that has substantially higher resolution than currently available devices and yet provides a depth of image of that is of high utility for medical diagnosticians.

### SUMMARY

These and other needs are addressed by the present disclosure which is directed towards providing an ultrasound imaging system that is portable. Such a system may be directly attached to the surface of the body and provide high resolution images of targeted subsurface body tissues with image resolution far superior to that of known state of the art ultrasound instruments without the need for a large, fixed and cumbersome instrument as required by other imaging technologies such as X-ray or MRI. Targeted tissues may include but not be limited to joints, ocular structures, and internal organs.

The present disclosure is also directed towards providing an ultrasound imaging system that can produce high image resolution (down to about 100 μm) at depths in the range of about 50 mm to about 60 mm. A number of known techniques are combined in a novel fashion and facilitated by an ultrasound system such as described in prior patents that stabilize the body part (or eye in some specific cases) relative to the ultrasound probe. Where the highest precision is required, a means of further elimination of movement due to breathing or heart beat is also disclosed.

An ultrasound scanner system is disclosed, comprising (1) an instrument body; (2) a linear positioner assembly interconnected to the instrument body; (3) an ultrasound probe which emits and receives an ultrasound pulse, the ultrasound probe interconnected to the linear positioner assembly wherein the ultrasound probe may operate in at least one dimension; (4) an instrument chamber disposed within the body and engaged with a membrane; and (5) a sealing chamber engaged with the membrane and a scanned object, wherein a transducer in the ultrasound probe emits and receives the ultrasound pulse through the instrument chamber, the membrane and the sealing chamber, wherein the ultrasound pulse contacts the scanned object.

A method is disclosed, comprising (1) sealing an ultrasound scanner to a scanned object; (2) filling a sealing chamber in a body of the ultrasound scanner with a sealing chamber fluid to provide a low acoustic impedance interface between the ultrasound scanner and a scanned object surface; (3) emitting and receiving an ultrasound pulse from a transducer contained in an ultrasound probe to generate an ultrasound image of a target matter within the scanned object.

The following definitions are used herein:

An arc scanner is a scanning device where the sensor moves in an precise arc about the center of the area to be scanned with its beam constantly directed through a central point.

Depth of focus is the distance over which the image plane can be displaced while a single object plane remains in acceptably sharp focus. The depth of focus is substantially symmetrical about the image plane when the image plane is at the focal distance

The focal length or focal distance of a focused ultrasound system is the distance between the transducer element (which emits an ultrasound pulse from a finite diameter element) and the point where the ultrasound beam diameter is a minimum and generally of maximum amplitude. The beam at this minimum diameter is said to be in focus.

MRI is magnetic resonance imaging.

Ocular as used herein means having to do with the eye or eyeball.

Ophthalmology as used herein means the branch of medicine that deals with the eye.

Optical as used herein refers to processes that use light rays.

Purkinje images are reflections of objects from structure of the eye. There are at least four Purkinje images that are visible on looking at an eye. The first Purkinje image (P1) is the reflection from the outer surface of the cornea. The second Purkinje image (P2) is the reflection from the inner surface of the cornea. The third Purkinje image (P3) is the reflection from the outer (anterior) surface of the lens. The fourth Purkinje image (P4) is the reflection from the inner (posterior) surface of the lens. Unlike the others, P4 is an inverted image. The first and fourth Purkinje images are used by some eye trackers, devices to measure the position of an eye. Purkinje images are named after Czech anatomist Jan Evangelista Purkyn\check{e} (1787-1869).

Sector scanner is an ultrasonic scanner that sweeps out a sector like a radar. The swept area is pie-shaped with its central point typically located near the face of the ultrasound transducer.

A specular surface as used herein means a mirror-like surface that reflects either optical or acoustic waves. For example, an ultrasound beam emanating from a transducer will only be reflected directly back to that transducer when the beam is aligned perpendicular to a specular surface.

Ultrasound means sound that is above the ear's upper frequency limit. When used for imaging object like the eye, the sound passes through a liquid medium, and its frequency is many orders of magnitude greater than can be detected by the ear. For high-resolution acoustic imaging in the eye, the frequency is typically in the approximate range of about 5 to about 80 MHz.

Ultrasound probe means an assembly comprised of a transducer element (typically a piezoelectric material), a probe body and electrical conduits that carry transmitted and received signals from the element to an A/D converter external to the probe.

Ultrasound pulse means a group of ultrasound waves centered around a center frequency where the pulse is comprised of at least one and up to about ten wave cycles. The ultrasound pulse is therefore a short burst of one to about ten wavelengths truncated at both ends of the wave train. An ultrasound pulse is further described in “Ultrasonography of the Eye and Orbit”, Second Edition, Coleman et al, published by Lippincott Williams & Wilkins, 2006 which is incorporated herein by reference.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### BRIEF DESCRIPTION OF THE DRAWINGS

The invention may take form in various components and arrangements of components, and in various steps and arrangements of steps. The drawings are only for purposes of illustrating some embodiments and are not to be construed as limiting the invention.

FIG. 1 is a side view of an ultrasound scanner system with a single element ultrasound transducer.

FIG. 2 is a top view of an ultrasound scanner system.

FIG. 3 is a side view of an ultrasound scanner system with an annular array ultrasound transducer.

FIG. 4 is a side view of an ultrasound scanner system with a linear array of ultrasound transducers.

To assist in the understanding of an embodiment of the present invention the following list of components and associated numbering found in the drawings is provided herein:

| Reference Number | Description |
|---|---|
| 1 | Instrument Housing |
| 2 | Conforming Body Seal |
| 3 | Ultrasound Probe |
| 4 | Positioning Eyepiece |
| 5 | Transparent Membrane |
| 6 | Saline Fluid Bag |
| 7 | Saline Fluid Fill Line |
| 8 | Instrument Fluid Fill Line |
| 9 | Signal and Control Cable Bundle |
| 10 | Linear Positioner Assembly |
| 11 | Instrument Chamber |
| 12 | Sealing Chamber |
| 14 | Patient Body Surface |
| 15 | Patient Body Part to be Scanned |
| 16 | Conforming Sealing Assembly |
| 17 | Motion and Pressure Sensor |
| 18 | Linear Array Probe |
| 21 | Ultrasound Probe Focal Distance |
| 22 | Probe Focal Plane |
| 23 | Ultrasound Probe Focal Distance |
| 24 | Maximum Distance Probe Tip to Membrane |
| 25 | Minimum Distance Probe Tip to Membrane |
| 26 | Maximum Distance Linear Array to Membrane |
| 27 | Minimum Distance Linear Array to Membrane |
| 28 | One Half Focal Depth |
| 51 | Ultrasound Probe x-Motion |
| 52 | Ultrasound Probe y-Motion |
| Mx | x-Position Motor and Actuator |
| My | y-Position Motor and Actuator |
| Mz | z-Position Motor and Actuator |

It should be understood that the drawings are not necessarily to scale. In certain instances, details that are not necessary for an understanding of the invention or that render other details difficult to perceive may have been omitted. It should be understood, of course, that the invention is not necessarily limited to the particular embodiments illustrated herein.

### DETAILED DESCRIPTION

The current disclosure is directed towards an imaging system that provides the convenience and lower cost of ultrasound imaging systems but with both high penetration depths and excellent resolution. This is achieved through a combination of existing ultrasound technologies and new methods for holding and stabilizing the ultrasound probe relative to the body surface and the body part to be imaged.

A conforming body seal and portable instrument body are disclosed that together provide for a compact yet stable fixation of the probe relative to the body surface as compared to currently available handheld ultrasound systems thereby enabling much longer scan times can be accommodated due to the elimination of probe movement relative to the body part to be imaged during the scan. With longer scan times advanced, ultrasound imaging techniques such as coded excitation (chirp excitation in its simplest form), over-sampling/averaging and dynamic focal plane imaging can be utilized allowing much higher operating frequencies (frequencies in a range of about 20 MHz to about 80 MHz) with their higher resolution and yet approaching penetration depths of traditional low frequency (about 5 MHz to about 10 MHz) ultrasound handheld systems

This arrangement includes computer controlled movement of the ultrasound probe providing multiple image cross-sections for 3D imaging of targeted tissue or selection of a particular cross-section from a set that best isolates the tissue of interest without having to reposition the system by hand multiple times. This arrangement provides much higher repeatability of image biometry due to repeatable fixation of the probe relative to the body surface.

### Portable Ultrasound Scanner

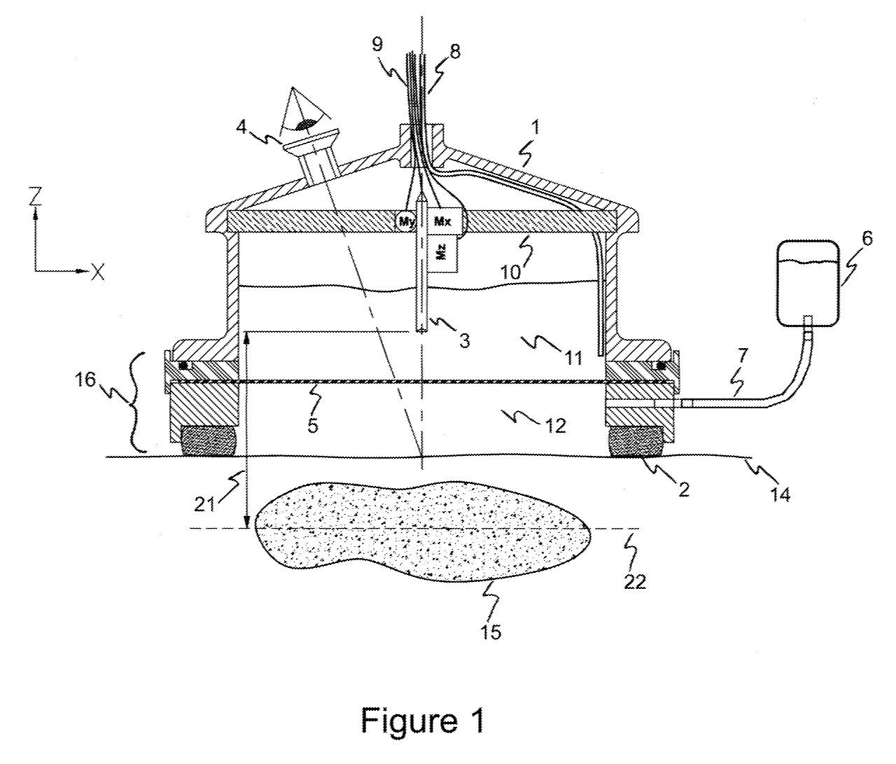

FIG. 1 is a side view illustrating the basic components of an ultrasound scanner system with an ultrasound probe comprised of a single element ultrasound transducer. The device is comprised of a housing assembly 1 which is pressed to the surface layer 14 of the patient overlaying the body part of interest 15 and sealed by a conforming seal 2 to minimize leakage of saline solution 12. A membrane 5 separates the saline solution 12 from the water 11 used to immerse the ultrasound probe 3. This membrane prevents water used in the probe section of the housing from mixing with the saline solution used in the section of the housing in contact with the patient.

Assembly 16 which holds membrane 5 in place is comprised of a clamp, sealing system and a conforming seal 2. Conforming seal 2 may also be comprised of a layer of adhesive to facilitate the seal adhering to the patient's skin 14. Assembly 16 corresponds in function to the disposable eyepiece used in an ultrasound eye scanner such as described in U.S. patent application Ser. No. 12/347,674, entitled “Components for an Ultrasonic Arc Scanning Apparatus” which is incorporated herein by reference. The entire assembly 16 is may be a disposable item that can be readily changed for each new patient.

As can be appreciated, the saline solution may or may not be a sterile saline solution depending on the body part to be scanned. If the body part to be scanned is covered by intact skin then the saline solution need not be sterile and may be replaced with distilled water. In some cases, only the conforming seal may be replaced for scanning other body parts. In general, the entire assembly 16 is replaced for each new patient. When the body part is an eye or when the body part is covered by injured or damaged skin, then a sterile saline solution should be used and the entire assembly should be replaced for each new patient.

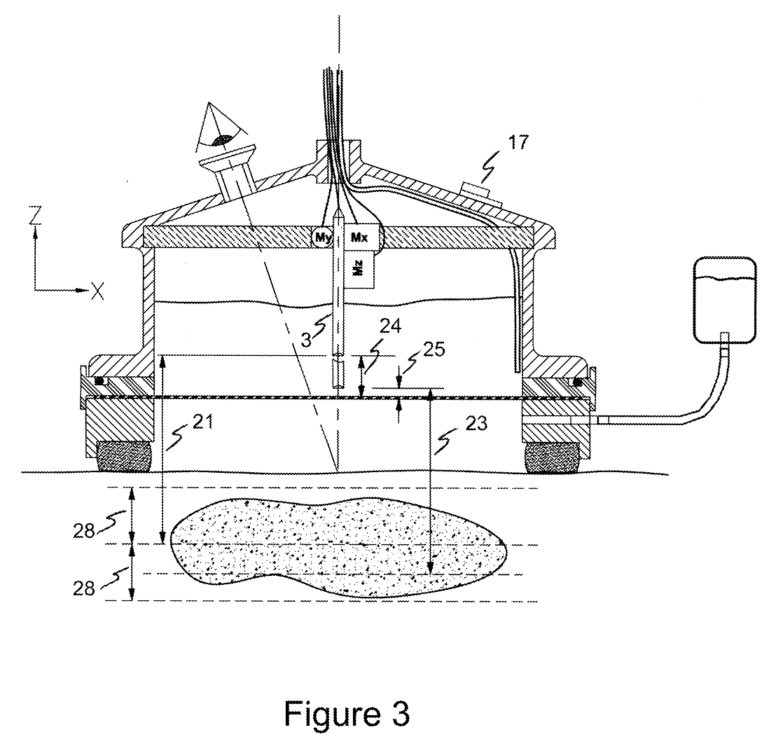

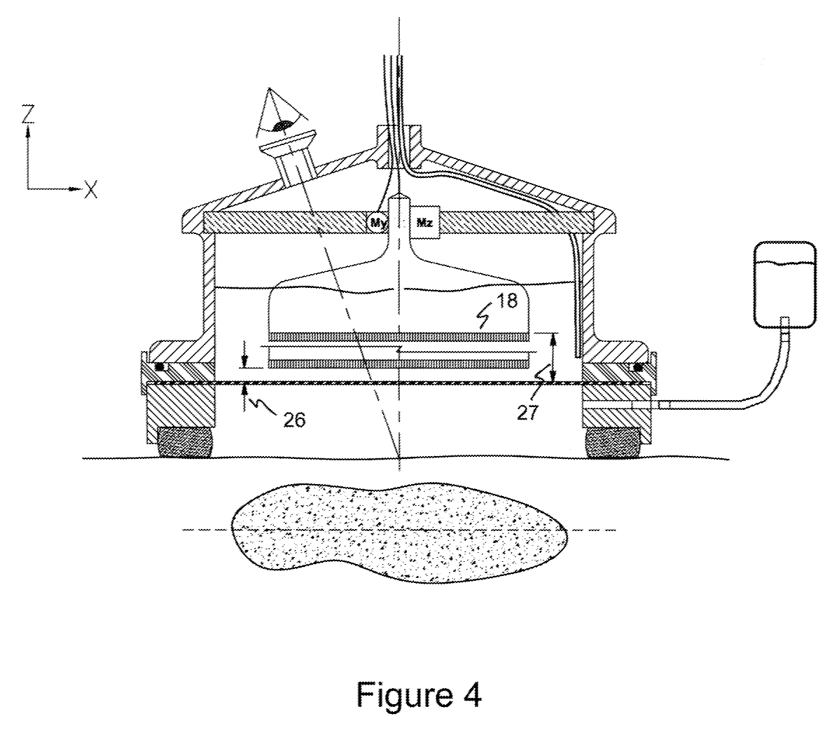

Both the saline solution 12 and water bath 11 are at ambient pressure, typically 1 atmosphere. In operation, the assembly is first placed over the body part of interest. Then the instrument chamber in the upper part of the housing is filled with water (typically distilled water) via fill tube 8, fully immersing the ultrasound probe 3. Then the saline solution 12 is introduced via fill tube 7 into the sealing chamber that connects a disposable saline fill bag 6. The operator can view the region around surface layer 14 overlaying the body part of interest 15 through positioning eyepiece 4. The motion of the ultrasound probe 3 is controlled in 3 orthogonal directions x, y and z by motors Mx, My and Mz. The operating instructions for these motors and the ultrasound probe 3 are communicated through cable bundle 9. Motors Mx, My and Mz are operated under computer control to move the probe up and down in the z-direction or back and forth in the x- and y-directions on linear positioning assembly 10. The nominal focal distance of the ultrasound transducer 21 is typically set as the distance from the pulse emitting element located at the tip of ultrasound probe 3 to approximately the centerline 22 of the body part of interest 15. The ultrasound probe may be comprised of a single pulse emitting and receiving element or a more complex probe embodiment such as described in FIGS. 3 and 4. If an ultrasound probe comprised of a single pulse emitting and receiving element is used, the center pulse frequency is typically in the range of about 5 MHz to about 80 MHz. The depth of field of a single frequency focused probe is typically about 1 to 2 millimeters at about 40 MHz and a focal distance of about 12 millimeters.

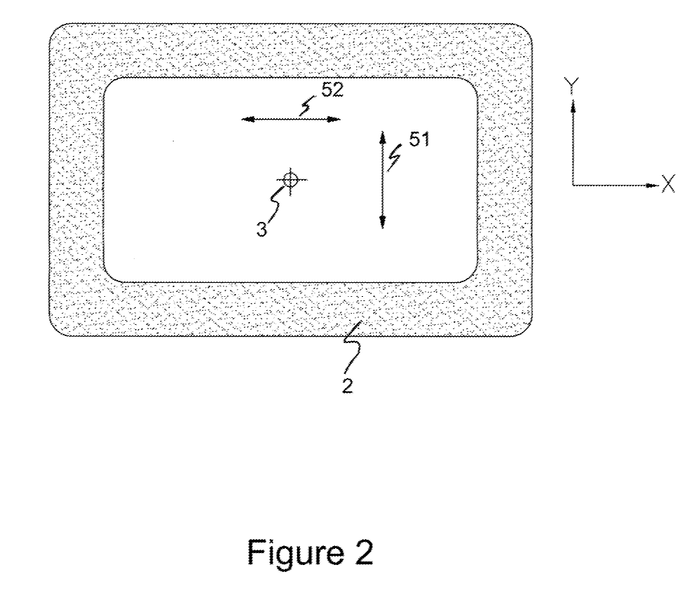

FIG. 2 is a top view of an ultrasound scanner system. This view shows the “footprint” of the conforming seal 2. Ultrasound probe tip 3 can move in the x-direction 52 and/or in the y-direction 51 by using the linear positioning assembly 10 described in FIG. 1.

An example of a linear positioning assembly is disclosed in U.S. patent application Ser. No.12/638,661, entitled “Alignment and Imaging of An Eye with an Ultrasonic Scanner” which is incorporated herein by reference.

### Description of Components

### Disposable Conforming Body Seal

The disposable body seal 16 is a component that may come in different shapes and forms to allow for optimal fit to a variety of body surfaces. It may have a shape to fit around the eye socket, be more curved to fit around a particular joint, or be more gently curved to fit on a more planar body surface such as an abdomen or back. All the sub-components described below are attached to one another into a single assembly 16.

A first sub-component is a conforming seal 2 of a low durometer plastic or foam which easily conforms to the body surface and outlines the entire area to be scanned. This seal is designed to adhere on the body side to the skin surface to provide a stable fixation of the system to the body and provide a seal of the sterile saline solution 12.

A second sub-component is a lower ring which provides for a rigid backing for the conforming seal and fill and drain lines 7 for the saline fill.

A third sub-component is a membrane 5 that separates the disposable saline fill from the instrument system fluid (which is typically distilled water and is filtered and may be reuseable). This membrane 5 must be acoustically transparent (that is, it must have an acoustic impedance similar to that of water) to the ultrasound beam and optically transparent to allow manual positioning of the system to the healthcare provider's mark on the body surface. This membrane is bonded to both the upper and lower rings and provides a fluid seal for both the handheld fluid on one side and the sterile saline solution on the other.

A fourth sub-component is an upper ring which provides for mechanical coupling of the body seal to the portable body and sealing of the fluid by O-ring or other suitable sealing method.

A fifth sub-component is a saline fill bag 6 and lines 7 which provide the single use sterile saline fill fluid. Fill control can be, for example, by squeezing the fill bag 6 by hand to force saline fluid into the volume between the membrane and body surface and out again through an overfill line.

### Instrument Main Body

The portable instrument main body 1 is a component that serves as the main frame of the system and serves as the mounting of several system components including the disposable body seal on the bottom of the instrument. The system components housed in the instrument main body include the positioner carriage (described below).

The portable instrument main body is comprised of a sealed chamber to contain the fluid (called the instrument chamber), typically distilled water, around the ultrasound probe. The system also includes fluid management system (not shown) which provides for filling and draining of the fluid 11 to/from the instrument chamber and for the fluids storage and maintenance. The system instrument main body also includes a feed-through to provide for a means to get the electrical signals to and from the ultrasound probe and the positioner motors and sensors, which are inside the instrument chamber, to an external pulser, motion control, signal/image processing and display system (described below). If the device is an eye scanner, then a fixation target is included which provides a means for aligning the visual axis of the eye to the ultrasound axis as disclosed in U.S. patent application Ser. No. 12/418,392, entitled “Procedures for an Ultrasonic Arc Scanning Apparatus” which is incorporated herein by reference.

The system also includes a positioning eyepiece (described below) and a means for strapping the handheld instrument (not shown) around the limb or torso or head as a backup to the body seal disposable to assist in the stabilization of the handheld instrument to the body surface.

### Positioner Carriage

The positioner assembly 10 is a subsystem that provides for fixating the ultrasound probe to a single or multi-axis movement system (including its actuators such as motors) which is in turn are mounted inside the sealed chamber of the instrument main body.

The movement system can be a single axis in its simplest form providing an image with only a single cross-section of the object to scan. The movement system could provide 2 or 3 axes of movement. In the system shown in FIG. 1 two linear axes of movement are shown in the x- and y-directions. This would provide for a multitude of cross-sections of an object with the possibility of providing 3D images. A third linear z-axis movement is shown for additional control of the depth of the optimal image for relatively deeper or shallower objects.

A movement axis could also be arcuate to better conform to the tissue to be imaged such as the cornea (as in the case of an eye scanner) or for a bending joint such as a knee or elbow.

### Positioning Eyepiece

The positioning eyepiece 4 is a simple, low power optical microscope allowing the operator to center the position of the instrument on a mark on the body surface placed prior to mounting of the instrument by the healthcare professional or in the case of the eye scanner centering on the iris of the eye. This insures the image will capture the selected body part. In the case of 2 or more positioning axes, this positioning need only be approximate as the scan area will be sufficient to allow complete capture of the object body part even if not perfectly centered on the object body part. In the case of the eye scanner, additionally a reflection of a fixation target will be centered through the eyepiece for precision alignment of the positioner carriage.

### Ultrasound Probe

An ultrasound probe 3 is a device that provides for delivering ultrasound pulse trains to the targeted object and receiving of the returning ultrasound echos, as is common practice in current ultrasound imaging systems. In FIG. 1, the probe is a cylindrical device and can either be a single or multiple element probe. Principal frequency is designed to be sufficiently high to allow much higher resolution than currently available handheld ultrasound imaging systems which are typically in the range of about 5 MHz to about 10 MHz. It is envisioned in this embodiment that the operating frequency of the probe be in the range of about 20 MHz to about 80 MHz. The focal plane of the probe is positioned at the approximate center plane of the targeted object. Advanced techniques for multiple-element annular elements are potentially used to allow for dynamic adjustment of the focal plane providing for multiple images with varying focal planes, then blended together for optimum full depth images across the entire thickness of the targeted object. Further, advanced coded excitation of the probe is used (such as chirp excitation and/or over-sampling) to further improve signal-to-noise ratio of the images particularly in the deepest parts of the scan where signal attenuation is at its highest. These advanced techniques will be important to achieving deeper imaging capability using a high frequency probe as the attenuation of the ultrasound signals in tissue are generally a function of frequency squared. It is highly desirable to use the higher operating frequency as the image resolution will increase linearly with operating frequency and thus providing much higher diagnostic value. This probe could also be a linear array and/or combined with a pivot mechanism at the probe's distal end to allow for pointing of the ultrasound beam from the end of the probe for more flexibility in optimizing scan geometry for best interface detection as disclosed in U.S. patent application Ser. No. 12/475,322, entitled “Compound Scanning Head for an Ultrasonic Scanning Apparatus which is incorporated herein by reference.

### Control, Processing and Display System

The external pulser, motion control, signal/image processing and display system (not shown) is a system may be configured as a standalone unit on a cart or nearby table or in a more compact form attached to the portable unit itself. This system provides the means for the following functions.

A first function is to produce the excitation signals to the ultrasound probe (the pulser). These excitation signals may be for a single element or for multiple elements discussed in FIG. 3 for an annular array probe embodiment or in discussed in FIG. 4 for a linear array embodiment. Also coded excitation may be utilized for improvements in signal to noise (along with their companion compression filters in the signal processing).

A second function is to carry out all signal processing of the returning ultrasound echoes including but not limited to such things as basics amplification and filtering of raw incoming signals, A/D conversion, all digital signal processing techniques, such as Fourier conversion and filtering, compression filtering and dynamic focusing.

A third function is to carry out imaging processing to convert the processed signals into images for viewing by the healthcare professional.

A fourth function is to provide the motioning control signals to the positioner actuators.

A fifth function is to provide for storage of processed ultrasound signals and their companion images for later retrieval.

A sixth function is to provide for the storage, filtering, delivery and evacuation of the instrument fluid that surrounds the ultrasound probe during scanning

### Operation

The following is a brief description of instrument operation. A new disposable body seal is attached to the bottom of the portable instrument main body. The center of the scanning area is marked by the healthcare professional on the patient's body. The adhesive on the bottom surface of the conforming seal is exposed and the portable instrument and body seal disposable assembly are carefully positioned on the patient's skin. The positioning eyepiece is used to center the instrument on the mark on the patient's skin provided by the healthcare professional while the body seal is pressed against the body surface. In the case of the eye scanner the instrument center is aligned to the iris of the eye. Optionally a handheld stabilizing strap may be used to wrap around the torso, head or limb of choice to further stabilize the portable unit. The cable between the portable instrument and external pulser, motion control, signal/image processing and display system is then attached. Thereupon, the handheld fluid filling operation is performed by turning on the fill pump on the unit thereby transporting the instrument fluid from the reservoir to the instrument chamber. The saline fill bag attached to the disposable sealing assembly is squeezed until fluid flows from the overfill tube to the sealing chamber.

The scanning parameters on the external pulser, motion control, signal/image processing and display system are then selected. In the case of the eye scanner, a second alignment will be required by centering the Purkinje reflection of the fixation target on cornea to the center of the instrument using the eyepiece and the positioner, thereby aligning the ultrasound axis to the visual axis of the eye as disclosed in U.S. patent application Ser. No. 12/418,392. The patient is instructed to stay focused on the fixation target throughout the scan. When the aforementioned steps are completed, the scan sequence is initiated. The scan sequence time is typically about a second to about several seconds. The images are reviewed to determine whether a re-scan is necessary. If the scans are acceptable, the procedure is terminated by draining and discarding the saline fluid from the sealing chamber. The instrument fluid is then pumped from the instrument chamber back to the reservoir. The body seal and stabilizing strap, if used, are removed from the patient. Finally, the disposable body seal is removed from portable unit and discarded.

### Annular Array Embodiment

The current disclosure is also directed towards an imaging system that provides the convenience and lower cost of ultrasound imaging systems but also with high penetration depths and resolution. This is achieved through a combination of existing ultrasound technologies and new methods for holding and stabilizing the ultrasound probe relative to the body surface.

One of the existing ultrasound technologies, annular array transducers, is described, for example, in the following two references. The first is entitled “Design and Fabrication of a 40-MHz Annular Array Transducer” by Jeffrey A. Ketterling, Orlando Aristiz'abal, Daniel H. Turnbull and Frederic L. Lizzi and is taken from IEEE Transactions on Ultrasonics, Ferroelectrics, and Frequency Control, Vol. 52, No. 4, April 2005. The second is entitled “Operational Verification of a 40-MHz Annular Array Transducer” by Jeffrey A. Ketterling, Sarayu Ramachandran and Orlando Aristiz'abal and is taken from IEEE Transactions on Ultrasonics, Ferroelectrics, and Frequency Control, Vol. 53, No. 3, March 2006.

FIG. 3 is a side view of an ultrasound scanner system with an annular array ultrasound transducer and illustrates the movement of an ultrasound annular array probe relative to body parts for extending the depth of high resolution imaging. FIG. 3 is comprised of the same components as those of FIG. 1 except that single transducer element ultrasound probe 3 is now a multi transducer element annular array probe. In addition, a solid state motion sensor 17 is shown attached to the outer housing. Such sensors can measure one or more of 3 axes of acceleration, 3 axes of gyroscopic (rotational) motion, 3 axes of gravitational variation and ambient pressure. The motion sensor may be used to compensate for the effects of breathing and/or heartbeat on the relative motion between the device housing and the body part being imaged. When ultrasound probe 3 is in a position with tip distance to the membrane 24, its focal center length is shown as length 21 and its depth of field is 2 times distance 28. For an annular array probe, the total depth of field may be approximately 5 to 6 millimeters at a center frequency of about 40 MHz. When ultrasound probe 3 is in a position with tip distance to the membrane 25, its focal center length is shown as length 23 and its depth of field is the same as before but centered at the new focal plane. Typically, focal length 21 and focal length 23 are the same.

As described below, the total high resolution image zone available by moving the ultrasound probe from tip distance to the membrane 24 (“D1”) to tip distance to the membrane 25 (“D2”), Z-total is then computed by the formula

Z-total=(D1−D2)+2Δz

where Δz (distance 28) is equal to half of the depth of field.

As noted previously, an conforming body seal and portable instrument body are disclosed that together provide for a compact yet stable fixation of the probe relative to the body surface and body part to be imaged as compared to prior art handheld ultrasound systems.

FIG. 3 illustrates the movement of an ultrasound annular array probe relative to body parts for extending the range of high resolution imaging. The elements of FIG. 3 include the following components.

The first is a z-axis positioner that allows rapid and precise setting of the distance of the ultrasound probe or array (single element, annular array or linear array) from the body surface. The z-axis actuator, Mz, is mounted on the positioner carriage. In this figure, the ultrasound probe is moved between positions D1 and D2 relative to the fluid separation membrane. The probe would be prevented from further z-axis motion past the membrane by a stop mechanism on the z-axis actuator.

The second is a y-axis positioner that allows for rapid and precise movement of the probe in the y-axis that allows for the creation of a B-Scan image.

A third is an ultrasound probe. In the case of an annular array, the depth of focus is increased through synthetic focusing to provide a much larger high resolution image zone of 2Δz. This high resolution zone is a multiple of the high resolution zone of a non-synthetically focused probe as is well known to those versed in the art of ultrasound imaging.

In operation, multiple sweeps of the probe in the y-axis are taken, each with a different probe z-position. Positions D1 and D2 are auspiciously chosen so there is some overlap of the high resolution zone. These zones can be combined with known windowing techniques.

The total high resolution image zone, Z-total is then computed by the formula

Z-total=(D1−D2)+2Δz

It is clear that z-total can be further extended by y-axis subsequent sweeps of the ultrasound probe. However, ultimately signal to noise will degrade as the high resolution zone goes deeper into the tissue due to absorption of the ultrasound signals as it passes through a longer path length in tissue. To overcome this, well-known techniques such as coded excitation (chirp excitation as a common example) and over-sampling will be required to image tissue at ever lower signal levels in the deepest tissue.

It must be noted that above disclosed techniques all require additional scanning time. In a prior art handheld device, this becomes problematic as hand motion interferes with proper imaging. However, probe stabilization as described above provides the stabilization of the probe and time needed to optimize the combination of techniques disclosed without the limits of scanning time imposed by other handheld devices.

In the case where the best possible image quality is needed and that even breathing or heart beating produces movement that would blur the image, a solid state gyroscope and/or accelerometer could be attached to the body of the handheld device to detect these biorhythms and then make subsequent scan sweeps during the same time of each rhythm thereby eliminating this source of motion noise. Alternately, these data can be used to compensate for these biorhythms in the signal processing step.

### Linear Array Embodiment

FIG. 4 is a side view of an ultrasound scanner system with a linear array ultrasound transducer and illustrates the movement of an ultrasound linear array relative to body parts for extending the depth of high resolution imaging. FIG. 4 is comprised of the same components as those of FIG. 1 except that the single transducer element probe or annular array probe is replaced by a 1D, 1½D or 2D linear array 18. In addition, a solid state motion sensor such as described in FIG. 3 may be included. The linear array may be used to change focal plane location and provide beam steering capability as described below. In the simplest embodiment, the linear array may be fixed in position. In other embodiments, the linear array assembly may be moved vertically in the z-direction and/or tilted about its center point and/or moved back and forth in the x-direction and y-direction for enhanced coverage.

FIG. 4 illustrates how a linear array can be used for enhancing Z-total by the ability to be moved in the z-direction. This method could be used for eye scanning as well as scanning body parts where only a z-axis motion is necessary for enhancing high resolution image depth. Further, a phased array could also be used to synthetically angle the beam to allow best signal to noise of reflections off of key biologic interfaces that are not normal to the straight on beam or to place the effective focal plane a various depths.

By using a linear phased array in the device shown in FIG. 4, it is possible to eliminate the positioner for scanning as it would only be required for the positioning step of centering on the Purkinje reflection in case of eye or the technician's reference mark on the body surface. The linear sweep could be duplicated with a linear array. In addition, by utilizing synthetic focusing, the outgoing beam could be tilted (using only the extreme 4-8 elements in the array as is commonly done) to obtain the effect of the pivoting head on the compound probe. Thus, we can make the desired measurements just using planar sweeps and beam tilting.

### Computer Control of the Positioning and Scanning Operations

In the above-described embodiments, some components of the scanner system may utilize computer controls, processing, or storage. The first actuator Mx, second actuator My, and third actuator Mz may require controls imputing for moving or articulating ultrasound probe 3 or linear 18. Further, the probe 3 or array 182 may require control inputs as well as processing and storage from a computer system.

In yet another embodiment, the disclosed systems and methods may be partially implemented in software that can be stored on a storage medium to include a computer-readable medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

In one embodiment, one or more computers are used to control, among other things, the rate or volume of dry product through one or more meters. In one embodiment, a user selectively inputs a volume or rate of one or more dry products through or into one or more meters. In one embodiment, the user interacts with the computer through any means known to those skilled in the art, to include a keyboard and/or display to include a touch-screen display. The term “computer-readable medium” as used herein refers to any tangible storage and/or transmission medium that participate in providing instructions to a processor for execution. Such a medium may take many forms, including but not limited to, non-volatile media, volatile media, and transmission media. Non-volatile media includes, for example, NVRAM, or magnetic or optical disks. Volatile media includes dynamic memory, such as main memory. Common forms of computer-readable media include, for example, a floppy disk, a flexible disk, hard disk, magnetic tape, or any other magnetic medium, magneto-optical medium, a CD-ROM, any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure is considered to include a tangible storage medium or distribution medium and prior art-recognized equivalents and successor media, in which the software implementations of the present disclosure are stored.

A number of variations and modifications of the inventions can be used. As will be appreciated, it would be possible to provide for some features of the inventions without providing others. For example, though the embodiments are discussed with reference to an arc scanning device, it is to be understood that the various embodiments may be used with other types of acoustic scanning devices using different transducer motion strategies.

The present invention, in various embodiments, includes components, methods, processes, systems and/or apparatus substantially as depicted and described herein, including various embodiments, sub-combinations, and subsets thereof. Those of skill in the art will understand how to make and use the present invention after understanding the present disclosure. The present invention, in various embodiments, includes providing devices and processes in the absence of items not depicted and/or described herein or in various embodiments hereof, including in the absence of such items as may have been used in previous devices or processes, for example for improving performance, achieving ease and\or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover though the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. For example, the steps may be performed in any order and are not limited to the particular ordering discussed herein.

What is claimed:

1. An ultrasound scanner system, comprising:
an instrument body;
a linear positioner assembly interconnected to the body;
an ultrasound probe which emits and receives an ultrasound pulse, the ultrasound probe interconnected to the linear positioner assembly wherein the ultrasound probe may operate in at least one dimension;
an instrument chamber disposed within the body and engaged with a membrane; and
a sealing chamber engaged with the membrane and a scanned object;
wherein one or more transducers in the ultrasound probe emits and receives the ultrasound pulse through the instrument chamber, the membrane and the sealing chamber, wherein the ultrasound pulse contacts the scanned object.

2. The system of claim 1, wherein the instrument chamber fluid is water.

3. The system of claim 1, wherein the sealing chamber fluid is a saline solution.

4. The system of claim 1, wherein the linear positioner assembly further comprises a plurality of actuators to allow three-dimensional movements.

5. The system of claim 1, wherein the instrument chamber further comprises an upper ring, the upper ring securing the membrane.

6. The system of claim 1, wherein the instrument chamber is in fluid communication with a fluid reservoir of instrument chamber fluid.

7. The system of claim 1, wherein the sealing chamber further comprises a lower ring, the lower ring securing the membrane.

8. The system of claim 1, wherein the sealing chamber is in fluid communication with a fluid reservoir of sealing chamber fluid.

9. The system of claim 7, wherein the sealing chamber further comprises a seal interconnected to the lower ring, the seal providing a seal between the ultrasound scanner and the scanned object surface.

10. The system of claim 1, wherein the body further comprises a viewing port such that an operator can discern a point on the scanned object surface, the point directly below the transducer.

11. The system of claim 1, wherein the instrument body further comprises at least one of a motion sensor and a pressure sensor.

12. The system of claim 1 where the ultrasound probe is comprised of one of a single element transducer, an annular array transducer and a linear array of transducers.

13. A method, comprising:
sealing an ultrasound scanner to a scanned object;
filling a sealing chamber in a body of the ultrasound scanner with a sealing chamber fluid to provide a low acoustic impedance interface between the ultrasound scanner and a scanned object surface;
emitting and receiving an ultrasound pulse from a transducer contained in an ultrasound probe to generate an ultrasound image of a target matter within the scanned object.

14. The method of claim 13, further comprising:
positioning the ultrasound scanner above a point on the scanner object surface, wherein a viewing port on the ultrasound scanner enables a user to view the point on the scanner object surface, the point directly below the ultrasound probe.

15. The method of claim 13, further comprising:
filling an instrument chamber in the ultrasound scanner with an instrument chamber fluid to provide a fluid environment for the ultrasound probe to emit and receive an ultrasound pulse through the instrument chamber fluid, a membrane, the sealing chamber fluid, and into the target matter with the scanned object.

16. The method of claim 15, wherein the instrument chamber is in fluid communication with an instrument chamber reservoir, the fluid communication through a communication port in the body of the ultrasound scanner.

17. The method of claim 13, wherein the sealing chamber is engaged with a lower ring, and the sealing chamber is in fluid communication with a sealing chamber reservoir, the fluid communication through a lower ring communication port in the lower ring.

18. The method of claim 13, further comprising:
strapping the ultrasound scanner to the scanned object to provide further support in addition to sealing the ultrasound scanner to the scanned object.

19. The method of claim 13, wherein the ultrasound probe is interconnected to at least one actuator, the actuator translating position in space of the ultrasound probe.

20. The method of claim 13 where the ultrasound probe is comprised of one of a single element transducer, an annular array transducer and a linear array of transducers.
